(19) United States
(12) Patent Application Publication
(10) Pub. No.: US 2003/0121520 A1
(43) Pub. Date: Jul. 3, 2003

(54) Mouthpiece to prevent air leakage and method for using the same

(76) Inventors: Lisa M. Parker, St. Joseph, MO (US); Sandra K. Sneathen, Cape Girardeau, MO (US)

Correspondence Address: STINSON MORRISON HECKER LLP, ATTN: PATENT GROUP, 1201 WALNUT STREET, SUITE 2800, KANSAS CITY, MO, 64106-2150, US

(21) Appl. No.: 10/034,782

(22) Filed: Dec. 27, 2001

(51) Int. Cl. A61M 16/00

(52) U.S. Cl. 128/206.21; 128/206.29

(57) ABSTRACT

The present invention is directed to a mouthpiece to be used in association with nCPAP systems to prevent leakage from the mouth during nCPAP treatment. The mouthpiece is comprised of a thin flexible disk that fits between the user's lips and teeth and seals the mouth around the interior of the lips to prevent air from escaping. The mouthpiece does not need to be custom-fitted by to the user. In a preferred embodiment, the mouthpiece of the present invention further comprises a bite block, affixed to the interior surface of the disk, configured to position the user's lower jaw to open the user's airway. In a most preferred embodiment, the disk of the mouthpiece of the present invention contains a one-way valve which allows the user to breathe air in through the disk, but prevents air from flowing out through the disk when the user exhales.

### CROSS-REFERENCE TO RELATED APPLICATIONS

Not Applicable

### STATEMENT REGARDING FEDERALLY SPONSORED RESEARCH OR DEVELOPMENT

Not Applicable.

### BACKGROUND OF THE INVENTION

1. Field of the Invention

The present invention is related to the field of airway management, more specifically to a device to prevent air leakage from the mouth.

2. Description of Related Art

Sleep apnea is a sleep disorder in which breathing stops for a matter of seconds or as long as two minutes. Treatments for sleep apnea depend on the severity of the condition, and range from changing sleep habits for less severe cases, to surgery in more severe cases. Compliance with treatment programs is a common problem with most treatment programs.

One of the most-widely used treatment methods for severe sleep apnea is continuous positive airflow pressure (CPAP), in which a machine supplies a steady stream of air through a tube that connects to a plastic mask. The plastic mask can cover both the mouth and nose, but usually covers only the nose (nCPAP). The machine provides sufficient air pressure to prevent the tissues in the user's airway from collapsing during sleep. The nCPAP treatment may be administered in a sleep laboratory. Alternatively, the user may be treated at home using a bedside nCPAP machine. Users should use nCPAP indefinitely to prevent recurrence of symptoms, unless and until the specific cause of the user's sleep apnea can be resolved.

The first step in nCPAP treatment is a titration process, performed in a sleep laboratory, to determine the amount of air pressure from the nCPAP system that is required to prevent the airway tissues from collapsing. It is important to accurately titrate the amount of pressure needed by the user during sustained treatment. Insufficient pressure will not adequately treat the problem. Too much pressure can be uncomfortable to the user, who may find it hard to exhale. Further, too much pressure can cause damage to the epithelial cells in the nasal passage, cause ulcers in the nose and mouth, and result in diseases of the mouth. Unfortunately, in nCPAP systems it is often difficult to perform an accurate titration to determine the amount of pressure needed, insofar as some of the air is released through the mouth. Mouth leakage generally results in a prescribed amount of pressure that is greater than needed by the user.

After the amount of pressure has been determined in the titration process, the nCPAP system may be used in sustained nCPAP treatment. During sustained treatment, mouth leakage is thought to cause high uni-directional nasal air flow, which may cause nasal congestion, dry nose and throat and sore throat.

Generally, compliance with the prescribed nCPAP program is low due to the negative effects of too much pressure and mouth leaks, the inconvenience of the bulky machine and the uncomfortable mask. One attempt to address problems with nCPAP involves humidifying the inspired air to decrease nasal congestion, dry nose and throat and sore throat. Another such attempt is referred to as a bi-level CPAP (biPAP) system, wherein the pressure is increased during inspiration to make the work of breathing easier for the user. However, neither of these adjustments fully address the problems associated with nCPAP.

To treat sleep apnea in mild to moderate cases, dental devices are commonly used. For example, common devices hold the tongue in a specific position to keep the patent's airway open. Other devices force the lower jaw forward, which keeps the airway open. Many such devices are similar to sports mouthpieces that cover the user's teeth. Such mouthpieces must generally be custom-fitted to the user's teeth in a time-consuming and costly process performed by a dental professional. While dental devices have shown to improve sleep and reduce snoring, it is unclear whether such devices treat the underlying sleep apnea. In addition, while compliance with programs for using such devices is greater than with the nCPAP system, many of the dental devices are formed of hard plastic and are therefore not comfortable. Further, unless the device is custom-fitted, most devices will not fit comfortably within the mouth.

### BRIEF SUMMARY OF THE INVENTION

The present invention is directed to a mouthpiece to be used in association with nCPAP systems to prevent leakage from the mouth during nCPAP treatment. The mouthpiece is comprised of a thin flexible disk that fits between the user's lips and teeth and seals the mouth around the interior of the lips to prevent air from escaping. Preferably the disk is made of a material the produces a slight adhesion with the inside of the user's lips and cheeks to assist in holding the mouthpiece in place. When used with an nCPAP system, the mouthpiece of the present invention seals the mouth to prevent air from the nCPAP system from leaking out of the mouth, allowing accurate titration of the air pressure needed by the user. Accurate titration using the mouthpiece of the present invention generally results in a lower prescribed pressure, which decreases the side effects of the nCPAP system, makes it easier for the user to exhale and, as a result, leads to increased compliance with the nCPAP treatment program. The mouthpiece may also be used by the user with home nCPAP systems to assure the air is traveling through the airway, and not out of the mouth, to achieve maximum benefits from the nCPAP program.

In a preferred embodiment, the mouthpiece of the present invention further comprises a bite block, affixed to the interior surface of the disk. The bite block is not fitted to the user's teeth. Rather, the bite block is preferably configured to rest interior of the user's teeth, to force the lower jaw forward, thereby opening the airway. In a preferred embodiment the bite block is configured as an “L” or “T” shape. In a most preferred embodiment, the bite block is less than the width of 2 to 3 upper incisors, so as to present minimum discomfort to the user.

In a most preferred embodiment, the disk of the mouthpiece of the present invention contains a one-way valve. The valve allows the user to breathe air in through the disk, but prevents air from flowing out through the disk when the user exhales. The valve thus allows the user to breathe more normally and to obtain air through sources other than the CPAP nasal mask in the event the nCPAP systems fails to provide air through the nose.

The mouthpiece of the present invention does not need to be custom-fitted to the user. If the disk of the mouthpiece is too large for a particular user, the user may simply cut around the exterior of the disk to produce a size that fits comfortably between the user's teeth and lips. Further, the disk of the present invention is formed of a thin, flexible plastic, which is comfortable to the user, and produces only minimal interference with the teeth and tongue to further add to the comfort level. The increased comfort should lead to increased compliance with the nCPAP program by the user.

### BRIEF DESCRIPTION OF THE DRAWINGS

FIG. 1 depicts a rear plan view of the mouthpiece of the present invention.

FIG. 2 depicts a cross-sectional view of the mouthpiece of the present invention taken along line 2-2 of FIG. 1

FIG. 3 depicts a cross-sectional view of the mouthpiece of the present invention taken along line 3-3 of FIG. 1.

FIG. 4 depicts front plan view of a preferred embodiment the mouthpiece of the present invention.

FIG. 5 depicts a perspective view of the mouthpiece of FIG. 4.

### DETAILED DESCRIPTION OF PREFERRED EMBODIMENT

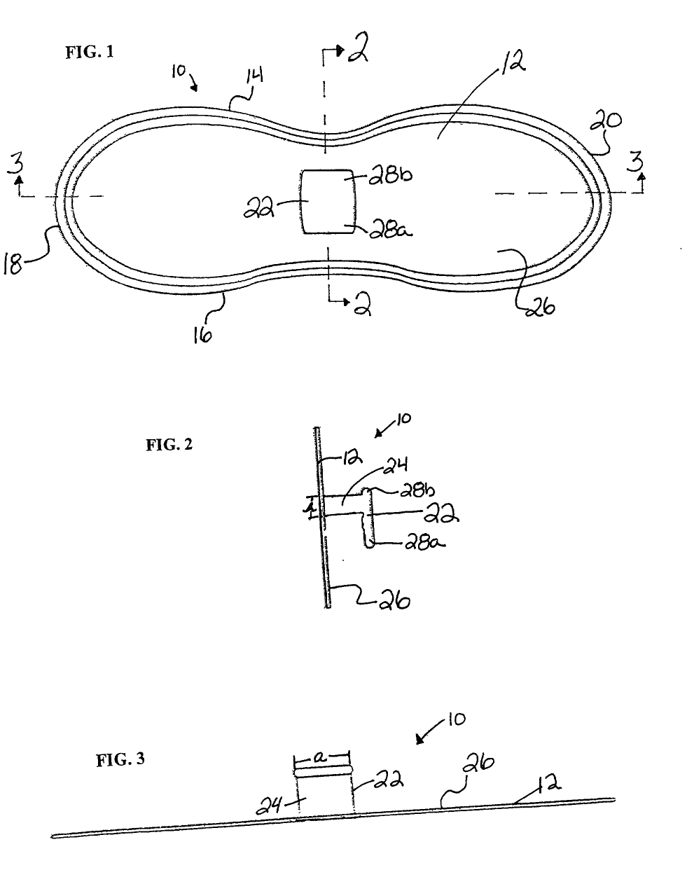

Turning to FIG. 1, the present invention is directed to mouthpiece 10 for use in sealing a user's mouth to prevent air from leaking out of the user's mouth. Mouthpiece 10 preferably comprises a thin flexible disk 12 that fits between the user's teeth and lips and seals the mouth around the interior of the lips and cheeks. Disk 12 is generally oval shaped, although other shapes that fit within the mouth and do not have sharp corners can be used consistent with the present invention. In a preferred embodiment, upper edge 14 and lower edge 16 of disk 12 are concave in shape, to prevent interference with the tissues projecting within the user's mouth.

Preferably, the width of disk 12, between first side edge 18 and second side edge 20 is slightly greater than the distance between the corners of the user's mouth opening. In a preferred embodiment the mouthpiece is approximately 100 to 150 mm wide, more preferably 145 mm wide. At its tallest point, the height of disk 12 between upper edge 14 and lower edge 16 is slightly greater than the distance between the base of the user's upper teeth and the base of the user's lower teeth when the user's mouth is partially closed. Preferably disk 12 is 45 to 70 mm high, more preferably 45 mm high. Disk 12 may be trimmed by the user to accommodate the size and shape of the user's mouth, provided disk 12 remains wide enough and high enough to seal the user's mouth opening. It should be understood that when used herein, the term “seal” does not require an airtight seal, but rather indicates that the exterior surface of mouthpiece 10 is positioned against the interior of the user's lips to prevent air from leaking out of the mouth. In a preferred embodiment, a portion of the exterior surface of mouthpiece 10 adheres to the interior of the user's lips. Most preferably, mouthpiece 10 produces an air-tight seal around the interior of the user's lips.

Disk 12 is preferably comprised of a thin flexible material. Because disk 12 is thin and flexible, it can easily be inserted in the user's mouth and remain in the user's mouth with a minimum amount of discomfort. Disk 12 is preferably less than 1.5 mm thick, more preferably less than 1.0 mm thick, most preferably less than 0.5 mm thick.

Disk 12 is preferably comprised of a material that provides a slight adhesion with the interior of the user's lips and cheeks to aid in sealing mouthpiece 10 within the user's mouth. Disk 12 is preferably comprised of a polymer, more preferably polyurethane, acrylic or silicone, most preferably silicone. Although disk 12 may be formed using any method known in the art or hereafter developed, it is preferably formed by injection molding.

In a preferred embodiment of the invention, mouthpiece 10 additionally comprises bite block 22, one preferred embodiment of which is shown in FIGS. 1, 2 and 3. When disk 12 is inserted between the user's teeth and lips, bite block 22 extends past the user's teeth, into the user's mouth. Preferably bite block 22 extends only a small distance past the user's teeth. In a preferred embodiment, bite block 22 extends 9 to 13 mm, more preferably 10 to 12 mm, and most preferably 11 mm from the interior surface 26 of disk 12.

In a preferred embodiment, bite block 22 is configured to maintain the user's lower jaw in a position that keeps the user's airway more open than when the lower jaw is in its natural resting position. For example, when the user's lower jaw is positioned slightly lower than and/or forward from it's natural resting position, user's airway is opened further than when the lower jaw is in its natural resting position. Reference to an “open airway” herein, refers to the condition wherein the user's airway is open further than when the user's lower jaw is it its natural resting position.

As best shown in FIGS. 2 and 3, bite block 22 preferably comprises an arm 24, generally perpendicular to the interior surface 26 of disk 12, with one or more flanges 28 extending generally perpendicular to arm 24. In a preferred embodiment, bite block 22 comprises downward flange 28a extending downwardly from the end of arm 24. In a most preferred embodiment, bite block 22 comprises downward flange 28a, extending downwardly from arm 24 and upward flange 28b, extending upwardly from arm 24. Alternatively, bite block 22 has no flanges 28, but rather a raised or indented notch is formed on the underside of arm 24 to maintain the user's lower teeth in a position forward of their natural resting position.

When inserted into a user's mouth, flange 24 extends between the user's upper and lower teeth, separating the teeth and maintaining the user's jaw slightly lower than its position when the user's mouth is fully closed. By positioning the user's jaw lower than its natural resting position, arm 24 maintains an open airway for the user.

Bite block 22 is preferably configured such that flanges 28 rest behind the user's teeth, a short distance from interior surface 26 of mouthpiece 10. Flanges 28 cause the user's lower teeth to rest slightly forward in the user's mouth, thereby positioning the user's jaw slightly forward from a natural resting position and maintaining an open airway. Further, bite block 22 of the present invention is configured to be used in a wide variety of mouths and need not be fitted to the user's teeth. As shown in FIG. 3, the height h of arm 24 is preferably greater than 5 mm, more preferably greater than 10 mm high. The width a of bite block 22 is preferably no wider than 2 or 3 of the user's upper incisors, and is preferably less than or equal to about 15 mm wide.

Bite block 22 may be comprised of any of the materials described with respect to disk 12, or may be comprised of a less flexible material. In any case, bite block 22 is comprised of a material strong enough not to collapse under pressure from the user's teeth. Most preferably bite block 22 is comprised of polycarbonate or acrylic. Bite block 22 may be formed using any method known in the art, such as injection molding, and may be affixed to disk 12 using any method known in the art, such as heat fusion.

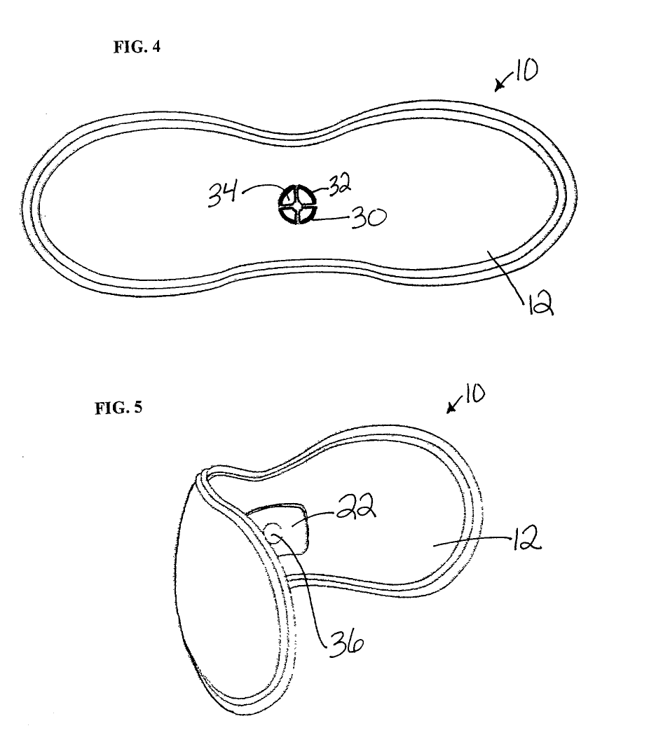

In a most preferred embodiment of the present invention best shown in FIG. 4, disk 12 of mouthpiece 10, contains one-way valve 30. One-way valve 30 is positioned to allow air to travel into the user's mouth, through disk 12, when the user inhales, but prevents air from traveling out of the user's mouth, through disk 12, when the user exhales. One-way valve 30 thus allows the user the comfort and safety of inhaling through disk 12, while preventing the user from exhaling through disk 12 in a manner would affect the effective positive air pressure provided by the nCPAP system.

One-way valve 30 may be any type of one-way valve known in the art, such as the type commonly used in CPR masks. Preferably one-way valve 30 comprises hard plastic frame 32, with thin flexible membrane 34 attached to frame 32 only at the center of frame 32. Alternatively, membrane 34 is comprised of a plurality of pie-shaped membrane pieces, affixed to the periphery of frame 32. Hard plastic frame 32 may be comprised of any hard plastic, preferably acrylic or polycarbonate. Membrane 34 is preferably less than 0.55 mm thick. Membrane 34 covers the lingual (interior) side of frame 32, such that air cannot be expelled through one-way valve 30, but can be inhaled through one-way valve 30 for the user's comfort, or in the event the nCPAP system malfunctions.

In the most preferred embodiment, mouthpiece 10 comprises both one-way valve 30 and bite block 22. In such embodiment, one-way valve 30 and bite block 22 are configured so as to allow air to be inhaled through one-way valve 30. As shown in FIG. 5, in such embodiment, bite block 22 is preferably formed with a hole 36 extending through flanges 28a and b and any portion of arm 24 blocking one-way valve 30. Hole 36 is positioned over one-way valve 30 to allow inhaled air to pass through one-way valve 30 and hole 36 in bite block 22. In such embodiment, bite block 22 and one-way valve 30 are preferably formed as a single piece. Alternatively, arm 24 of bite block 22 may be placed above or below one-way valve 30, such that bite block 22 does not block air from being inhaled through one-way valve 30, although flanges 28a and b may overhang one-way valve 30. In such embodiment arm 24 and one-way valve 30 may be formed as a single piece, with arm 24 extending from the upper or lower edge of valve 30, or may be formed as separate pieces.

From the foregoing it will be seen that this invention is one well adapted to attain all ends and objectives herein-above set forth, together with the other advantages which are obvious and which are inherent to the invention.

Since many possible embodiments may be made of the invention without departing from the scope thereof, is to be understood that all matters herein set forth or shown in the accompanying drawings are to be interpreted as illustrative, and not in a limiting sense.

While specific embodiments have been shown and discussed, various modifications may of course be made, and the invention is not limited to the specific forms or arrangement of parts and steps described herein, except insofar as such limitations are included in the following claims. Further, it will be understood that certain features and sub-combinations are of utility and may be employed without reference to other features and sub-combinations. This is contemplated by and is within the scope of the claims.

What is claimed and desired to be secured by Letters Patent is as follows:

1. A mouthpiece for preventing air leakage, comprising:
a flexible disk configured to fit between a user's teeth and lips and seal the user's mouth,
wherein the thickness of said disk is less than or substantially equal to 1.5 mm.

2. The mouthpiece of claim 1, wherein the thickness of said disk is less than or substantially equal to 1.0 mm.

3. The mouthpiece of claim 2, wherein the thickness of said disk is less than or substantially equal to 0.5 mm.

4. The mouthpiece of claim 1, wherein said disk is comprised of a material that naturally adheres to the interior of the user's lips and cheeks.

5. The mouthpiece of claim 4, wherein said disk is comprised of silicone.

6. The mouthpiece of claim 4, further comprising a bite block configured to position the user's lower jaw in a position that maintains an open airway.

7. The mouthpiece of claim 6, wherein said bite block comprises:
an arm extending substantially perpendicular to an interior surface of said disk; and
a flange extending substantially perpendicular to said arm.

8. The mouthpiece of claim 7, wherein the width of said arm and the width of said flange are less than or substantially equal to 15 mm.

9. The mouthpiece of claim 8, wherein said flange extends downward from said arm.

10. The mouthpiece of claim 4, further comprising a one-way valve in said disk configured to allow air to pass through said disk from an exterior of said disk to an interior of said disk.

11. The mouthpiece of claim 6, further comprising a one-way valve in said disk configured to allow air to pass through said disk from an exterior of said disk to an interior of said disk.

12. A mouthpiece for preventing air leakage, comprising:
a flexible disk configured to fit between a user's teeth and lips; and
a bite block extending from an interior surface of said disk, wherein said bite block comprises:
an arm extending substantially perpendicular to the interior surface of said disk; and
a flange extending substantially perpendicular to said arm, wherein the width of said arm and the width of said flange are less than or substantially equal to 15 mm.

13. The mouthpiece of claim 12, wherein the thickness of said disk is less than or substantially equal to 1.5 mm.

14. The mouthpiece of claim 12, wherein said disk is comprised of a material that naturally adheres to the interior of the user's lips and cheeks.

15. The mouthpiece of claim 12, further comprising a one-way valve in said disk configured to allow air to pass through said disk from an exterior of said disk to an interior of said disk.

16. A mouthpiece for preventing air leakage, comprising:
a flexible disk configured to fit between a user's teeth and lips, wherein said disk is comprised of a material that naturally adheres to the interior of the user's lips and cheeks; and
a one-way valve in said disk configured to allow air to pass through said disk from an exterior of said disk to an interior of said disk.

17. A method for preventing air leakage during nasal continuous positive airflow pressure treatment comprising:
applying a mask to cover the user's nose, wherein said mask is affixed to an air source; and
inserting a mouthpiece into the user's mouth, wherein said mouthpiece is configured to prevent air from leaking out of the user's mouth.

18. The method for preventing air leakage as claimed in claim 17, wherein said mouthpiece adheres to the interior of the user's lips.

19. The method for preventing air leakage as claimed in claim 17, wherein said mouthpiece comprises a bite block affixed to an interior surface of said mouthpiece, wherein said bite block is configured to position the user's lower jaw in a position that maintains an open airway.

20. The method for preventing air leakage as claimed in claim 19, wherein said bite block comprises:
an arm extending substantially perpendicular to the interior surface of said disk; and
a flange extending substantially perpendicular to said arm, wherein the width of said arm and the width of said flange are less than or substantially equal to 15 mm.

21. The method as claimed in claim 19, wherein said mouthpiece further comprises a one-way valve configured to allow air to pass through said mouthpiece from an exterior of said mouthpiece to the interior of the user's mouth.

22. The method as claimed in claim 17, wherein said mouthpiece further comprises a one-way valve configured to allow air to pass through said mouthpiece from an exterior of said mouthpiece to the interior of the user's mouth.
